(19) United States
(12) Patent Application Publication
(10) Pub. No.: US 2026/0358322 A1
(43) Pub. Date: Nov. 12, 2026

(54) ROBUST CALCULATION METHOD FOR SOIL HYDRAULIC PARAMETERS BASED ON ONE-DIMENSIONAL UPWARD INFILTRATION EXPERIMENT

(71) Applicant: Institute of Soil Science, Chinese Academy of Sciences, Nanjing (CN)

(72) Inventors: Donghao MA, Nanjing (CN); Sicong WU, Nanjing (CN); Jiabao ZHANG, Nanjing (CN); Yiqing CHEN, Nanjing (CN); Ziyan GUO, Nanjing (CN)

(73) Assignee: Institute of Soil Science, Chinese Academy of Sciences, Nanjing (CN)

(21) Appl. No.: 19/664,919

(22) Filed: Apr. 30, 2026

(30) Foreign Application Priority Data
May 9, 2025 (CN) 202510594431.3

(51) Int. Cl. G01N 33/24

(52) U.S. Cl. CPC G01N 33/246

(57) ABSTRACT

Provided is a robust calculation method for soil hydraulic parameters based on a one-dimensional upward infiltration experiment. The method constructs a soil hydraulic parameter inversion method based on the piston-type water profile model (PASCP), and effectively expands the applicability of the model by innovatively converting the non-uniform water content distribution of the unsaturated zone into an equivalent uniform zone. Based on this model, the method constructs an analytical infiltration model that accounts for a saturation time and introduces an interval-constrained optimization objective function, significantly improving the inversion accuracy and robustness of key hydraulic parameters of the Brooks-Corey model. Experiments demonstrate that the method exhibits excellent applicability under different soil textures, and particularly yields higher result reliability in the presence of measurement errors. The method provides an efficient and practical technical means for rapid and accurate characterization of soil hydraulic properties through a standard cutting ring-based parameter estimation method.

### TECHNICAL FIELD

The present disclosure relates to a robust calculation method for soil hydraulic parameters based on a one-dimensional upward infiltration experiment, and belongs to the technical field of soil physics and soil hydrology.

### BACKGROUND TECHNOLOGY

Soil hydraulic parameters, including the soil water retention curve (SWRC) θ(h) and unsaturated hydraulic conductivity curve K(θ), are key input parameters for hydrological models based on the Richards equation. Together, they determine the transport characteristics of soil water in the unsaturated zone. Accurate determination of these soil hydraulic parameters is not only crucial for water and solute transport studies at the field scale but also of great significance for hydrological and energy cycle simulations at the global scale. The conventional laboratory methods for determining θ(h) and K(θ) are the pressure membrane method and the instantaneous profile method, respectively. Though simple in theory, these methods involve cumbersome and time-consuming experimental procedures. To improve efficiency, numerical inversion techniques for the simultaneous estimation of θ(h) and K(θ) based on transient flow experiments have been rapidly developed, including single-/multi-step outflow experiments, evaporation methods, and upward infiltration methods. Among them, the upward infiltration inversion technique based on cutting ring soil samples has made important breakthroughs in the past decade. This method adopts the inverse-gravity infiltration process driven by capillary action, which features relatively slow water movement. It is particularly suitable for accurately measuring the water flux through a 5 cm high cutting ring, thereby providing an effective pathway for large-scale soil hydraulic survey. However, existing numerical inversion techniques still face a fundamental challenge, that is, there is a lack of universal and efficient algorithms for solving the highly nonlinear Richards equation. Current numerical schemes often require customized spatial discretization strategies for different soil types and boundary conditions. Such strong case dependence severely restricts their wider practical application.

To address the universality and complexity issues faced by the above numerical methods, researchers have gradually turned their attention to analytical and semi-analytical physical models, with the aim of more efficiently predicting soil hydraulic parameters. Physical models have also been applied to parameter inversion under upward infiltration conditions. Moret-Fernandez and Latorre (2016), based on the Haverkamp model and the saturated hydraulic conductivity Kₛ determined by Darcy's law, successfully retrieved the sorptivity S and the shape parameter β, and further estimated θ(h). Chinese Patent (CN114397427A) discloses a method for predicting soil hydraulic properties based on an upward infiltration process of a cutting ring soil sample, providing a novel algorithm that can simultaneously estimate θ(h) and S. However, both of the above methods require additional determination of the soil saturated hydraulic conductivity Kₛ as an input parameter to estimate θ(h). As pointed out by van Genuchten (1991), the proposer of the van Genuchten model as a classical soil hydraulic model, the Kₛ parameter is often difficult to measure accurately. To address this problem, Wu et al., based on the Chinese Patent (CN114397427A), proposed a physical model of constant-pressure upward infiltration, i.e. approximate solutions under constant pressure (ASCP), in a paper published in the Journal of Hydrology in 2022. This model can simultaneously estimate the key parameters describing θ(h) and K(θ), including the pore size distribution index n, the inverse of air-entry suction 1/h_{d}, and the saturated hydraulic conductivity Kₛ. However, the accurate estimation of parameters by the ASCP method relies on a strict equality constraint, i.e., the accurate determination of the single-point cumulative infiltration (I*) when the wetting front first reaches the soil surface and the corresponding soil column height (L*). However, in actual laboratory measurements, due to the limited accuracy of air pressure sensors and the disturbance of indoor air pressure by bubbles in the Mariotte bottle (Latorre et al., 2015), the I* parameter when the wetting front just reaches the soil surface is often difficult to measure accurately. This results in the failure to meet the strict equality constraint, thereby degrading the performance of the ASCP method under practical conditions. Therefore, reducing the impact of the measurement error on I′ and enhancing the robustness of the method has become a key issue that needs to be addressed urgently. Furthermore, experimental evidence from Moret-Fernandez and Latorre (2019) shows that after the wetting front reaches the soil surface, the soil water absorption process continues slowly until a final steady state is reached. Considering that the time required for different soil textures to reach a steady state varies significantly, the infiltration data during this stage may include additional key information about soil hydraulic properties. If such information can be incorporated into the ASCP method, it can further improve the accuracy of hydraulic parameter estimation.

### CONTENT OF THE INVENTION

To overcome the deficiencies of the prior art, an objective of the present disclosure is to provide a robust calculation method for soil hydraulic parameters based on a one-dimensional upward infiltration experiment, further improving the accuracy of hydraulic parameter estimation.

The present disclosure adopts the following technical solution.

A robust calculation method for soil hydraulic parameters based on a one-dimensional upward infiltration experiment includes following steps:
step A: applying a constant lower-boundary water head hₚ to a cutting ring soil sample, conducting a one-dimensional upward infiltration experiment on a homogeneous soil, and recording a relationship between a cumulative infiltration I of the cutting ring soil sample and a time t, a time t* when a wetting front first reaches a surface of the cutting ring soil sample, and a cumulative infiltration I_{end} when an infiltration curve tends to stabilize;
step B: calculating a saturated volumetric water content θₛ of the soil based on an initial volumetric water content θᵢ of the cutting ring soil sample, a height L* of a cutting ring, and the cumulative infiltration I_{end} when the soil reaches complete saturation:

$${θ}_{s} = {θ}_{i} + \frac{{I}_{end}}{{L}^{*}} ; \qquad ( 13 )$$

step C: determining, based on the relationship between the cumulative infiltration I and the time t obtained in the step A, a single-point cumulative infiltration I* at the time t* when the wetting front first reaches the surface of the cutting ring soil sample; and
step D: obtaining, by an interval-constrained parameter optimization-based inversion method, parameters to be estimated: n, 1/h_{d}, and Kₛ, according to the saturated volumetric water content θₛ, the initial volumetric water content θᵢ, and a residual volumetric water content θᵣ of the cutting ring soil sample, the single-point cumulative infiltration I* when the wetting front first reaches the soil surface, and observed data I_{obs}(tᵢ) of the cumulative infiltration over the time.

Preferably, in the step D, an objective function for the interval-constrained parameter optimization-based inversion method is constructed according to Eq. (14):

$$minimize Φ = \sqrt{\frac{1}{N} {\sum }_{i = 1}^{N} {( {I}_{obs} ( {t}_{i} ) - {I}_{sim} ( {t}_{i} ; n , 1 / {h}_{d} , {K}_{s} ) )}^{2}} + σ max \{ 0 , \vert {I}_{sim} ( {L}^{*} ; n , 1 / {h}_{d} , {K}_{s} ) - {I}^{*} \vert - ε \}$$

Preferably, in a first term of Eq. (14), I_{obs}(tᵢ) is a measured value obtained in the step A, representing the cumulative infiltration I at an observation time point tᵢ; and Iₛᵢₘ(tᵢ; n, 1/h_{d}, Kₛ) denotes a simulated cumulative infiltration at the observation time point tᵢ, calculated according to following equations:
(1) a soil saturation time (tₛ) is expressed as:

$${t}_{s} = ( {θ}_{s} - {θ}_{i} ) \{ - \frac{{z}_{s}^{*}}{{K}_{s}} + [ \frac{{H}_{f}}{{K}_{e}} - \frac{{h}_{d} + {h}_{p}}{{K}_{s}} ] ln ( 1 - \frac{{z}_{s}^{*}}{{h}_{d} + {h}_{p}} ) - \frac{{H}_{f}}{{K}_{e}} ln ( 1 - \frac{{K}_{s} - {K}_{e}}{{K}_{s} ( {h}_{d} + {h}_{p} )} {z}_{s}^{*} ) + \frac{{H}_{f}}{{K}_{s} - {K}_{e}} ( \frac{1}{1 + \frac{{K}_{s} - {K}_{e}}{{K}_{s} ( {h}_{d} + {h}_{p} )} {z}_{s}^{*}} - 1 ) \}$$

where,

$${z}_{s}^{*}$$

denotes a functional expression of an equivalent wetting front z_{fe}; and when the equivalent wetting front z_{fe} reaches the soil surface (i.e., z_{fe}=L*), the soil reaches a saturated state, and

$${z}_{s}^{*}$$

is explicitly expressed as:

$${z}_{s}^{*} = \frac{{A}_{3} {L}^{*} + {A}_{4} + 1 - \sqrt{{( {A}_{3} {L}^{*} + {A}_{4} + 1 )}^{2} - 4 {A}_{3} {L}^{*}}}{2 {A}_{3}} \qquad ( 7 )$$

(2) when the infiltration time t is less than tₛ, a functional relationship between the cumulative infiltration I of the soil and the time tis:

$$\frac{t}{{θ}_{s} - {θ}_{i}} = - \frac{{h}_{d} + {h}_{p}}{{J}_{w 0} + {K}_{s}} + [ \frac{{H}_{f}}{{K}_{e}} - \frac{{h}_{d} + {h}_{p}}{{K}_{s}} ] ln ( 1 - \frac{{K}_{s}}{{J}_{w 0} + {K}_{s}} ) - \frac{{H}_{f}}{{K}_{e}} ln ( 1 - \frac{{K}_{s} - {K}_{e}}{{J}_{w 0} + {K}_{s}} ) + \frac{{H}_{f}}{{K}_{s} - {K}_{e}} ( \frac{1}{1 - \frac{{K}_{s} - {K}_{e}}{{J}_{w 0} + {K}_{s}}} - 1 ) \qquad ( 8 )$$

$$I = ( {θ}_{s} - {θ}_{i} ) [ \frac{{K}_{s} ( {h}_{d} + {h}_{p} )}{{J}_{w 0} + {K}_{s}} - \frac{{K}_{e} {H}_{f}}{{J}_{w 0} + {K}_{e}} ] \qquad ( 9 )$$

(3) when t is not less than tₛ, the soil reaches complete saturation, and the cumulative infiltration remains unchanged, and is calculated as follows:

I=(θₛ−θᵢ)L* (10);
substituting the parameters to be estimated, n, 1/h_{d}, Kₛ, into the equation enables a root mean square error (RMSE) between the measured value I_{obs}(tᵢ) and the simulated value Iₛᵢₘ(tᵢ; n, 1/h_{d}, Kₛ) to be minimized;
in a second term of Eq. (14), Iₛᵢₘ(L*; n, 1/h_{d}, Kₛ) denotes a cumulative infiltration obtained by simulation calculation, calculated as follows:

$$I = {A}_{0} {z}_{f} + \frac{( {θ}_{s} - {θ}_{i} - {A}_{0} )}{2 {A}_{1}} ( {A}_{1} {z}_{f} + {A}_{2} + 1 - \sqrt{{( {A}_{1} {z}_{f} + {A}_{2} + 1 )}^{2} - 4 {A}_{1} {z}_{f}} ) , \qquad ( 11 )$$

where, z_{f}=L*; σ denotes a penalty factor [-]; ϵ denotes a constraint threshold [mm], with a value depending on an upper limit of a maximum measurement error in actual observation;
remaining parameters are expressed as follows:

$$\{ \begin{matrix}{K}_{e} = ζ {K}_{s} = \frac{n ( a + 1 ) ( {θ}_{s} - {θ}_{i} )}{[ a ( 3 n + 2 ) + n ] [ {θ}_{s} - {θ}_{i} - a ( {θ}_{i} - {θ}_{r} ) ]} {K}_{s} \\ {H}_{f} = - \frac{1}{ζ ( 3 n + 1 )} {h}_{d} \\ {A}_{0} = ( {θ}_{s} - {θ}_{r} ) \frac{1 - ( 1 + a b ) {S}_{ei}}{b ( a + 1 )} \\ {A}_{1} = \frac{{K}_{s} - {K}_{e}}{{h}_{d} + {h}_{p}} \\ {A}_{2} = - \frac{{θ}_{s} - {θ}_{i}}{{A}_{0}} \frac{{K}_{e} {H}_{f}}{{K}_{s} ( {h}_{d} + {h}_{p} )} \\ {A}_{3} = - \frac{{K}_{s} - {K}_{e}}{{K}_{s} ( {h}_{d} + {h}_{p} )} \\ {A}_{4} = - \frac{{K}_{e} {H}_{f}}{{K}_{s} ( {h}_{d} + {h}_{p} )}\end{matrix} \qquad ( 12 )$$

Preferably, Eq. (14) is solved using a genetic algorithm (GA package) in R language: the algorithm generates 1000 sets of parameters to be estimated, n, 1/h_{d}, Kₛ, in each generation; the algorithm stops when φ in Eq. (14) does not improve for 500 consecutive generations or when a cumulative number of iterations reaches 5000; a local search strategy is enabled, where parameter search ranges are: Kₛ=[10⁻⁶, 10⁻²] cm·s⁻¹, 1/h_{d}=[0.01, 1] cm⁻¹, and n=[0.1, 0.7]; logarithmic transformation is performed on Kₛ and 1/h_{d}; the constraint threshold ε is set to 0.4 mm; and σ is set to \sqrt{.Machine$double.xmax}, where .Machine$double.xmax denotes a maximum double-precision floating-point number representable in R language.

The present disclosure proposes a novel robust calculation method for soil hydraulic parameters based on a one-dimensional upward infiltration experiment (PASCP method). Unlike the ASCP method, which can only describe the infiltration process before the wetting front reaches the soil surface, the PASCP method further expands the applicable scope. It incorporates soil hydraulic characteristic information included in the steady-state stage (i.e. the infiltration stage that continues after the wetting front reaches the soil surface) into parameter estimation, and realizes complete simulation of the entire upward infiltration process from the initial wetted state to complete saturation of the soil, significantly improving the accuracy of hydraulic parameter estimation. Furthermore, to reduce the impact of measurement errors on the measured value of the single-point cumulative infiltration I* when the wetting front first reaches the soil surface, the present disclosure proposes an interval-constrained parameter optimization method on the basis of the PASCP method to achieve robust simultaneous estimation of the soil hydraulic parameters (n, 1/h_{d}, and Kₛ) of the Brooks-Corey model.

The present disclosure has the following beneficial effects:
1. The PASCP method effectively overcomes the limitation in applicability of the ASCP method after the wetting front reaches the soil surface, providing an important novel approach for robust estimation of soil hydraulic parameters.
2. By adopting the interval-constrained parameter optimization-based inversion method, the present disclosure reduces the influence of measurement errors on the single-point cumulative infiltration amount (I*) when the wetting front first reaches the soil surface, significantly improving the robustness of soil hydraulic parameter estimation.
3. The standard cutting ring-based parameter estimation method provides an effective technical means for rapid and reliable characterization of soil hydraulic properties.

### DESCRIPTION OF THE DRAWINGS

FIG. 1 is a schematic diagram of the evolution of a soil water profile during a soil upward infiltration, where (a) shows a profile shape when a wetting front first reaches a soil surface; (b) shows a profile variation when the infiltration process continues after the wetting front reaches the surface; and (c) shows a profile shape when the soil reaches a completely saturated steady state;

FIG. 2 is a flowchart of calculation according to the present disclosure;

FIG. 3 shows an example of a parameter inversion experiment of a clay soil sample, where (a) is a schematic diagram of a one-dimensional upward infiltration experimental device and a measurement process for a laboratory cutting ring soil sample; (b) shows a measured cumulative infiltration curve and key time points, including a time (t*) when the wetting front first reaches the soil surface and a time (tₛ) when the soil reaches complete saturation; (c) shows iterative trajectories of PASCP and ASCP methods during parameter estimation; and (d) shows a comparison of SWRCs inverted by the PASCP and ASCP methods with measured values;

FIG. 4 is a parameter estimation diagram of five soils with significantly different textures measured by a laboratory experiment; and

FIG. 5 is a parameter estimation diagram of seven soils with significantly different textures determined based on numerical simulation, where (a) shows that the PASCP method incorporates infiltration information of a steady-state stage; and (b) shows that the PASCP method excludes infiltration information of the steady-state stage.

The present disclosure will be further described below with reference to specific embodiments.

### SPECIFIC IMPLEMENTATIONS

The content of the present disclosure is further described below in conjunction with a soil infiltration experiment, but this should not be construed as limiting the present disclosure. Modifications and substitutions made to methods, steps or conditions of the present disclosure without departing from the spirit and essence of the present disclosure fall within the scope of the present disclosure. Unless otherwise specified, the technical means used in the embodiments are conventional means well known to those skilled in the art.

### Embodiment 1: Derivation Process of Modified ASCP with Piston-Type Water Profile (PASCP) Method

### 1. Method for Constructing Piston-Type Water Profile

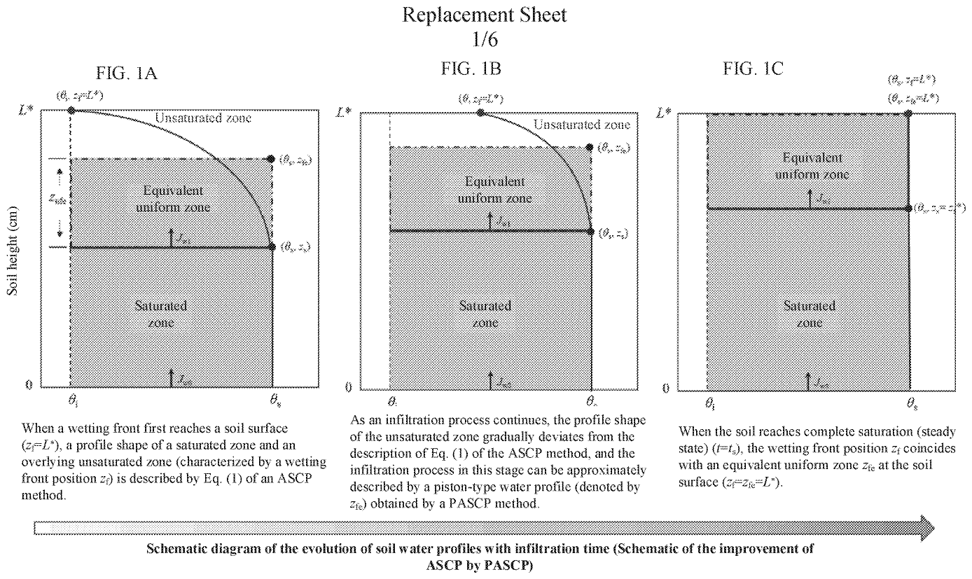

The soil hydraulic parameter inversion method based on the piston-type water profile model (PASCP) of the present disclosure is specifically an improvement and extension of the ASCP method earlier proposed by the applicant (Wu et al., 2022, Journal of Hydrology). In the ASCP method, the soil water profile includes a saturated zone and an unsaturated zone. The water distribution remains uniform in the saturated zone, whereas an obvious gradient variation occurs in the unsaturated zone (FIG. 1a). The mathematical expression of this profile is:

$${S}_{e} = \frac{θ - {θ}_{r}}{{θ}_{s} - {θ}_{r}} = \{ \begin{matrix}1 & 0 \leq z < {z}_{s} \\ {( 1 - b \frac{z - {z}_{s}}{{z}_{f} - {z}_{s}} )}^{a} & {z}_{s} < z \leq {z}_{f}\end{matrix} \qquad ( 1 )$$

where, z denotes a soil depth [cm], with its zero point set at the soil bottom and the positive direction of the coordinate axis upward; z_{f} denotes a wetting front advance distance [cm]; zₛ denotes a length of the saturated zone [cm]; Sₑᵢ denotes an effective saturation [-] corresponding to initial water content θᵢ; n denotes a pore size distribution index; and parameters a and b are defined as follows:

$$a = \frac{n ( 2 n + 3 )}{( 2 n + 2 ) ( 2 n + 1 )} \qquad ( 2 )$$

$$b = 1 - {S}_{ei}^{\frac{1}{a}} \qquad ( 3 )$$

However, the ASCP method can only describe the infiltration process before the wetting front reaches the soil surface. After the wetting front reaches the surface, the unsaturated zone gradually approaches complete saturation (FIG. 1), and the water profile deviates from the prediction of the ASCP equation (Eq. 1). To address this limitation, based on the piston flow assumption proposed by Green and Ampt (1911), the present disclosure develops a novel construction method for piston-type water profiles, namely the PASCP method. The innovation lies in avoiding explicit simulation of the gradual saturation process in the unsaturated zone, while accurately characterizing the cumulative infiltration during this stage, which effectively extends the applicable range of the ASCP method. Specifically, an integral method is adopted to convert the non-uniform water content distribution of the unsaturated zone into an equivalent uniform zone, and the equivalent length z_{ufe} of the unsaturated zone is defined, which is calculated as follows:

$${z}_{ufe} = \frac{{\int }_{{z}_{s}}^{{z}_{f}} ( θ - {θ}_{i} ) dz}{{θ}_{s} - {θ}_{i}} = \frac{1 - ( 1 + a b ) {S}_{ei}}{b ( a + 1 ) ( 1 - {S}_{ei} )} ( {z}_{f} - {z}_{s} ) \qquad ( 4 )$$

where, z_{ufe} denotes the equivalent length of the unsaturated zone, and zₛ denotes the length of the saturated zone. Summing zₛ and z_{ufe} yields the equivalent piston-type wetting front z_{fe}, which is expressed as:

z_{fe}=zₛ+z_{ufe} (5)

With the above piston-type water profile model adopted, the PASCP method can effectively simulate the upward advancement of the wetting front in a piston-type water profile mode. When the equivalent wetting front z_{fe} reaches the soil surface (as shown in FIG. 1c), the soil column becomes completely saturated and attains a steady state. This time is defined as the soil saturation time (tₛ) [min].

### 2. Derivation of Analytical Model

Using the above piston-type water profile, a one-dimensional upward infiltration analytical model suitable for a constant-water-head boundary condition is further derived based on the principle of mass conservation.
(1) The soil saturation time (tₛ) is expressed as:

$${t}_{s} = ( {θ}_{s} - {θ}_{i} ) \{ - \frac{{z}_{s}^{*}}{{K}_{e}} + [ \frac{{H}_{f}}{{K}_{e}} - \frac{{h}_{d} + {h}_{p}}{{K}_{s}} ] ln ( 1 - \frac{{z}_{s}^{*}}{{h}_{d} + {h}_{p}} ) - \frac{{H}_{f}}{{K}_{e}} ln ( 1 - \frac{{K}_{s} - {K}_{e}}{{K}_{s} ( {h}_{d} + {h}_{p} )} {z}_{s}^{*} ) + \frac{{H}_{f}}{{K}_{s} - {K}_{e}} ( \frac{1}{1 + \frac{{K}_{s} - {K}_{e}}{{K}_{s} ( {h}_{d} + {h}_{p} )} {z}_{s}^{*}} - 1 ) \} \qquad ( 6 )$$

where, zₛ* is a functional expression of the equivalent wetting front z_{fe}. When the equivalent wetting front z_{fe} reaches the soil surface (i.e., z_{fe}=L*), the soil attains a saturated state, and

$${z}_{s}^{*}$$

can be explicitly expressed as:

$${z}_{s}^{*} = \frac{{A}_{3} {L}^{*} + {A}_{4} + 1 - \sqrt{{( {A}_{3} {L}^{*} + {A}_{4} + 1 )}^{2} - 4 {A}_{3} {L}^{*}}}{2 {A}_{3}} \qquad ( 7 )$$

It is worth noting that tₛ depends on the parameters n, 1/h_{d}, and Kₛ, which indicates its application value in parameter inversion.
(2) When the infiltration time t is less than tₛ, a functional relationship between the cumulative infiltration I of the soil and the time tis:

$$\frac{t}{{θ}_{s} - {θ}_{i}} = - \frac{{h}_{d} + {h}_{p}}{{J}_{w 0} + {K}_{s}} + [ \frac{{H}_{f}}{{K}_{e}} - \frac{{h}_{d} + {h}_{p}}{{K}_{s}} ] ln ( 1 - \frac{{K}_{s}}{{J}_{w 0} + {K}_{s}} ) - \frac{{H}_{f}}{{K}_{e}} ln ( 1 - \frac{{K}_{s} - {K}_{e}}{{J}_{w 0} + {K}_{s}} ) + \frac{{H}_{f}}{{K}_{s} - {K}_{e}} ( \frac{1}{1 - \frac{{K}_{s} - {K}_{e}}{{J}_{w 0} + {K}_{s}}} - 1 ) \qquad ( 8 )$$

$$I = ( {θ}_{s} - {θ}_{i} ) [ \frac{{K}_{s} ( {h}_{d} + {h}_{p} )}{{J}_{w 0} + {K}_{s}} - \frac{{K}_{e} {H}_{f}}{{J}_{w 0} + {K}_{e}} ] \qquad ( 9 )$$

(3) When t is not less than tₛ, the soil reaches complete saturation, and the cumulative infiltration remains unchanged, and is calculated as follows:

I=(θₛ−θᵢ)L* (10)
(4) The functional relationship between the cumulative infiltration I and the wetting front advance distance z_{f} is:

$$I = {A}_{0} {z}_{f} + \frac{( {θ}_{s} - {θ}_{i} - {A}_{0} )}{2 {A}_{1}} ( {A}_{1} {z}_{f} + {A}_{2} + 1 - \sqrt{{( {A}_{1} {z}_{f} + {A}_{2} + 1 )}^{2} - 4 {A}_{1} {z}_{f}} ) \qquad ( 11 )$$

The definitions of the unknown parameters in the above equations are as follows: θᵢ denotes an initial volumetric water content of the soil [cm⁻³·cm⁻³], θₛ denotes a saturated volumetric water content of the soil [cm⁻³ cm⁻³], hₚ denotes a water supply head at a lower boundary of the soil [cm], and L* denotes a soil height [cm]. The remaining parameters are expressed according to the following relationships:

$$\{ \begin{matrix}{K}_{e} = ζ {K}_{s} = \frac{n ( a + 1 ) ( {θ}_{s} - {θ}_{i} )}{[ a ( 3 n + 2 ) + n ] [ {θ}_{s} - {θ}_{i} - a ( {θ}_{i} - {θ}_{r} ) ]} {K}_{s} \\ {H}_{f} = - \frac{1}{ζ ( 3 n + 1 )} {h}_{d} \\ {A}_{0} = ( {θ}_{s} - {θ}_{r} ) \frac{1 - ( 1 + a b ) {S}_{ei}}{b ( a + 1 )} \\ {A}_{1} = \frac{{K}_{s} - {K}_{e}}{{h}_{d} + {h}_{p}} \\ {A}_{2} = - \frac{{θ}_{s} - {θ}_{i}}{{A}_{0}} \frac{{K}_{e} {H}_{f}}{{K}_{s} ( {h}_{d} + {h}_{p} )} \\ {A}_{3} = - \frac{{K}_{s} - {K}_{e}}{{K}_{s} ( {h}_{d} + {h}_{p} )} \\ {A}_{4} = - \frac{{K}_{e} {H}_{f}}{{K}_{s} ( {h}_{d} + {h}_{p} )}\end{matrix} \qquad ( 12 )$$

The method for determining the residual volumetric water content θᵣ of the soil is as follows. The sieved soil is placed in a dry air environment with a relative humidity of less than 15% for several months until its mass remains constant. At this time, the water content of the soil is the residual mass water content of the soil, which is then multiplied by the bulk density of the cutting ring soil sample to obtain the residual volumetric water content of the soil. The initial volumetric water content θᵢ of the soil is determined by an oven-drying method. The saturated volumetric water content θₛ of the soil is determined by inverse calculation of Eq. (10), and its expression is:

$${θ}_{s} = {θ}_{i} + \frac{{I}_{end}}{{L}^{*}} \qquad ( 13 )$$

where, I_{end} denotes a stable value [cm] when the cumulative infiltration curve reaches a plateau, which represents the total amount of water absorbed by the soil sample.

### 3. Interval-Constrained Parameter Optimization-Based Inversion Method

The present disclosure further proposes an interval-constrained parameter optimization inversion method based on the PASCP method to improve the accuracy and robustness of soil hydraulic parameters (n, h_{d}, and Kₛ) estimation. The expression of the objective function of the specific algorithm is:

$$Φ = \sqrt{\frac{1}{N} {\sum }_{i = 1}^{N} {( {I}_{o b s} ( {t}_{i} ) - {I}_{sim} ( {t}_{i} ; n , 1 / {h}_{d} , {K}_{s} ) )}^{2}} + σ max \{ 0 , \vert {I}_{sim} ( {L}^{*} ; n , 1 / {h}_{d} , {K}_{s} ) - {I}^{*} \vert - ε \} \qquad ( 14 )$$

In the equation, the first term denotes a root mean square error (RMSE) between measured data I_{obs}(tᵢ) and simulated data Iₛᵢₘ(tᵢ; n, 1/h_{d}, Kₛ). The simulated value Iₛᵢₘ(tᵢ; n, 1/h_{d}, Kₛ) is calculated by Eqs. (6) to (10), where tᵢ denotes an observation time point, and Iₛᵢₘ denotes the cumulative soil infiltration at the corresponding time. The second term introduces an interval constraint through a penalty function to ensure that the deviation between the simulated cumulative infiltration Iₛᵢₘ(L*; n, 1/h_{d}, Kₛ) and the single-point cumulative infiltration I* when the wetting front first reaches the soil surface remains within [symbol] ε, so as to account for the impact of experimental measurement errors on inversion accuracy. In the equation, Iₛᵢₘ(L*; n, 1/h_{d}, Kₛ) is calculated by Eq. (11) (i.e., z_{f}=L*); σ denotes a penalty factor [-]; and ϵ denotes a constraint threshold [mm], and its value depends on the upper limit of the maximum measurement error in actual observations.

The above measured data (the relationship between the cumulative infiltration Ii and the time ti) can be obtained using a field portable device for the rapid determination of soil hydraulic properties disclosed by the applicant (Chinese Patent Application No.: CN216209116U). Constant water head hₚ is applied to the bottom of the cutting ring soil sample, with a measurement frequency of once per second. A camera is used to determine the exact time (t*) when the wetting front reaches the soil surface. From the measured data (I versus t), the cumulative infiltration I* at the time when the wetting front reaches the soil surface is obtained according to t*. The cumulative infiltration I_{end} when the infiltration curve tends to stabilize is recorded. The height L* of the cutting ring is measured.

Eq. (14) is solved using a genetic algorithm (GA package) in R language. The algorithm generates 1000 sets of parameters to be estimated, n, 1/h_{d}, and Kₛ, in each generation. It stops when the φ value in Eq. (14) no longer improves for 500 consecutive generations or when the cumulative number of iterations reaches 5000. A local search strategy is enabled. The parameter search ranges are: Kₛ=[10⁻⁶, 10⁻²] cm·s⁻¹, 1/h_{d}=[0.01, 1] cm⁻¹, and n=[0.1, 0.7]. Logarithmic transformation is performed on Kₛ and 1/h_{d}. The constraint threshold ε is set to 0.4 mm; and σ is set to \sqrt{.Machine$double.xmax}, where .Machine$double.xmax denotes the maximum double-precision floating-point number representable in R language.

### Embodiment 2

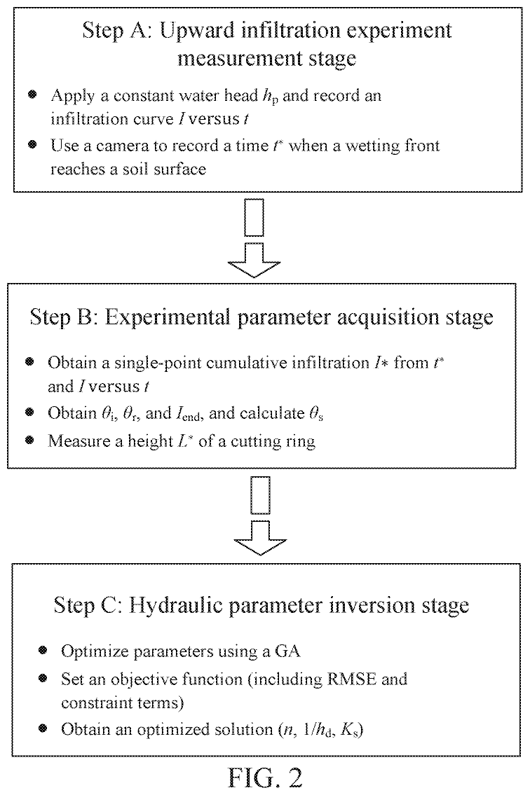

In this embodiment, a parameter inversion experiment is carried out based on the PASCP method (FIG. 2). The specific operations are as follows:

### Step A: Upward Infiltration Experiment Measurement Stage:

(1) The constant water head hₚ is applied to the bottom of the cutting ring soil sample.
(2) The relationship between the cumulative infiltration I of the soil sample and time t is recorded.
(3) A camera is used to record the time t′ when the wetting front first reaches the surface of the soil sample.

### Step B: Experimental Parameter Acquisition Stage:

(1) From the measured data, the cumulative infiltration I* when the wetting front reaches the soil surface is obtained according to t*.
(2) The initial water content θᵢ and residual water content θᵣ of the soil sample are determined.
(3) The cumulative infiltration I_{end} when the infiltration curve tends to stabilize is recorded, and the saturated water content θₛ is calculated.
(4) The height L* of the cutting ring is measured.

### Step C: Model Parameter Inversion Stage:

(1) An optimization objective function is constructed based on the GA in R language, including the RMSE term and the interval constraint term.
(2) The population size is set to 1000, the maximum number of iterations is set to 5000 (or 500 generations), and the local search strategy is enabled.
(3) The parameter search ranges are: Kₛ=[10⁻⁶, 10⁻²] cm·s⁻¹, 1/h_{d}=[0.01, 1] cm⁻¹, n=[0.1, 0.7]; and logarithmic transformation is performed on Kₛ and 1/h_{d}.
(4) The optimal estimated values of the soil hydraulic parameters n, 1/h_{d}, and Kₛ are obtained through optimization calculation.

The measured data in the step A (the relationship between the cumulative infiltration I and the cumulative time t, I_{obs}(tᵢ)) is obtained using the field portable device for rapid determination of soil hydraulic properties disclosed by the applicant (Chinese Patent Application No.: CN216209116U), with a measurement frequency of once per second.

In the step B, the method for determining the residual volumetric water content θᵣ of the soil is as follows. The sieved soil is placed in a dry air environment with a relative humidity of less than 15% for several months until its mass remains constant. At this time, the water content of the soil is the residual mass water content of the soil, which is then multiplied by the bulk density of the cutting ring soil sample to obtain the residual volumetric water content of the soil. The initial volumetric water content θᵢ of the soil is determined by an oven-drying method. The saturated volumetric water content θₛ of the soil is determined according to Eq. (13).

The objective function in the step C is specifically shown in Eq. (14), solved using the GA based on R language, with the population size set to 1000, and the algorithm stops after a maximum of 5000 iterations or 500 generations, provided that the fitness has not improved. The local search strategy is enabled, and the parameter search ranges are: Kₛ=[10⁻⁶, 10⁻²] cm·s⁻¹, 1/h_{d}=[0.01, 1] cm⁻¹, n=[0.1, 0.7]. Logarithmic transformation is performed on Kₛ and 1/h_{d}. The constraint threshold ε is set to 0.4 mm; and σ is set to \sqrt{.Machine$double.xmax}, where .Machine$double.xmax denotes the maximum double-precision floating-point number representable in R language. The simulated infiltration Iₛᵢₘ(L*; n, 1/h_{d}, Kₛ) in Eq. (14) is calculated according to Eq. (11), and the simulated data Iₛᵢₘ(tᵢ; n, 1/h_{d}, Kₛ) is calculated according to Eqs. (6) to (10).

### Embodiment 3

The purpose of this embodiment is to verify the ability of the PASCP method to handle measurement errors under the condition that there are errors in actual measurements, and to demonstrate the improvement in the robustness of parameter estimation compared to the ASCP method. According to the steps of Embodiment 2, the following specific experiment is conducted.

The collected soil sample is first air-dried and passed through a 2 mm sieve. The sieved soil is placed in a dry air environment with a relative humidity of less than 15% for several months until its mass remains constant. The residual mass water content of the soil is then determined by a gravimetric method. Based on the predetermined bulk density of 1.4, the residual volumetric water content θᵣ is calculated to be 0.025 cm³·cm⁻³. Subsequently, the soil sample is slowly packed layer by layer into a cutting ring with an inner diameter of 50.46 mm, a height of 50 mm, and a nylon mesh laid at the bottom. During the packing process, a uniform bulk density is achieved by gently tapping the cutting ring and performing interlayer roughening treatment. After the cutting ring is packed, the initial volumetric water content θᵢ of the soil in the cutting ring is 0.025 cm³·cm⁻³.

For the packed cutting ring, the cumulative infiltration curve (I versus t) is measured using the device for rapid determination of soil hydraulic properties, specifically with a constant water head hₚ=0 cm. Infiltration data are acquired once per second using the portable infiltration device, and a camera is used to record the time when the wetting front reaches the surface, t*=3356 s. The corresponding cumulative infiltration I* when the wetting front reaches the soil surface is 1.848 cm. The cumulative infiltration I_{end} when the infiltration curve tends to stabilize is recorded as 1.984 cm, and the saturated volumetric water content θₛ is calculated to be 0.422 cm³ cm⁻³. The height L′ of the cutting ring is measured to be 5 cm.

An optimization objective function (Eq. (14)) is constructed using the GA based on R language. Specifically, the population size is set to 1000, and the maximum number of iterations is set to 5000 (or 500 consecutive generations). The local search strategy is enabled, and the parameter search ranges are: Kₛ=[10⁻⁶, 10⁻²] cm·s⁻¹, 1/h_{d}=[0.01, 1] cm⁻¹, and n=[0.1, 0.7]. Logarithmic transformation is performed on Kₛ and 1/h_{d}. The known parameters θₛ, θᵢ, θᵣ, I*, L*, and hₚ, combined with Eqs. (6) to (11), are used to calculate Iₛᵢₘ(L*; n, 1/h_{d}, Kₛ) and Iₛᵢₘ(tᵢ; n, 1/h_{d}, Kₛ) in Eq. (14). The constraint threshold ε is set to 0.4 mm. Finally, the optimal estimated values of the soil hydraulic parameters n, 1/h_{d}, and Kₛ are obtained through optimization calculation according to Eq. (14).

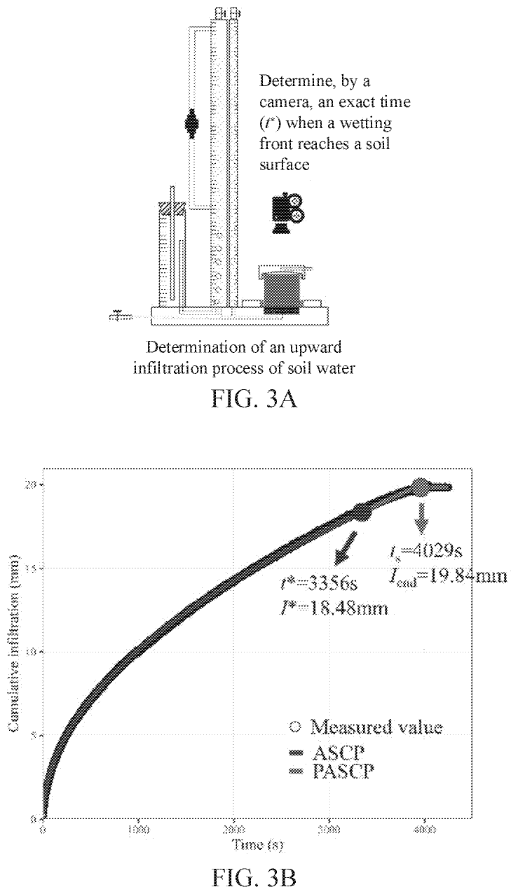

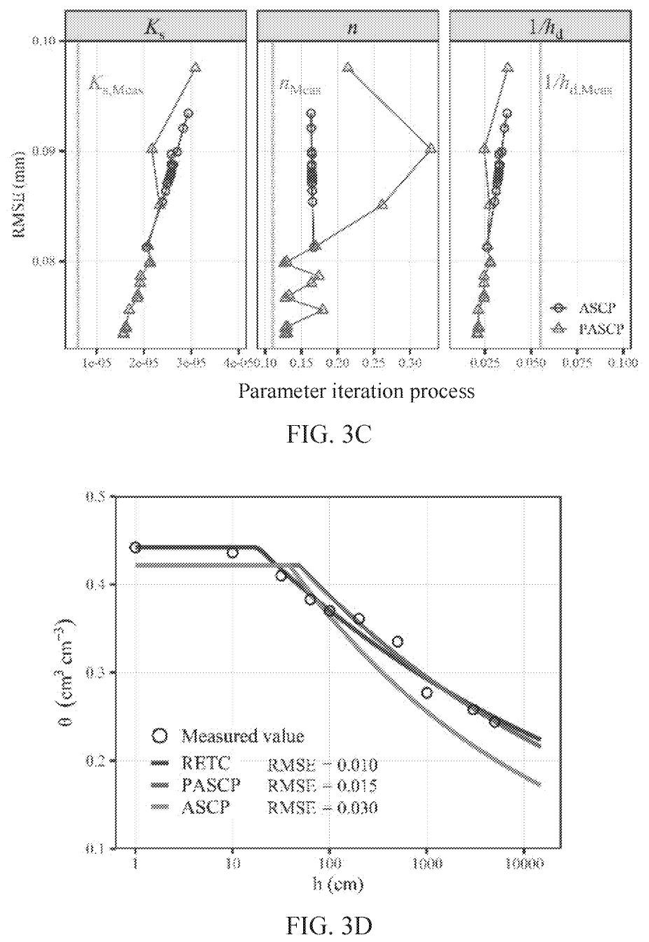

FIG. 3 shows the parameter iterative estimation processes of the PASCP and ASCP methods. The ASCP method is used as a comparative reference to evaluate the improvement effect of the PASCP method on the robustness of parameter estimation. FIG. 3a shows that the PASCP method has a wider parameter search range, while the search space of the ASCP method is limited due to its strict equality constraint. Interestingly, some of the parameter values finally determined by the ASCP method coincide with those searched during the iteration process of the PASCP method. This phenomenon indicates that the parameter search space of the PASCP method encompasses the search range of the ASCP method, highlighting the robustness of the PASCP method in the presence of large measurement errors. FIG. 3b further shows that the SWRC predicted by the PASCP method is in better agreement with the measured values (RETC), while the prediction of the ASCP method shows a significant deviation (RMSE_ASCP=0.030, RMSE_PASCP=0.015, RMSE_RETC=0.010).

### Embodiment 4

The purpose of this embodiment is to extensively verify the accuracy and parameter identification capability of the present disclosure in estimating soil hydraulic parameters. Five soils with significantly different textures are used as research objects (as shown in Table 1). The properties of the five soils include: soil texture composition (percentage contents of sand, silt, and clay), soil organic carbon content (SOC), bulk density (ρ_{b}), soil residual water content (θᵣ), soil saturated water content (θₛ), soil initial water content (θᵢ), and the single-point cumulative infiltration (I*) when the wetting front reaches the soil surface. The data in the table are measured values determined by standard methods, with reference to Klute (1986) for the measurement standards. A comparison is conducted with the ASCP method to highlight the advantages of the present disclosure. Since the specific measurement steps for the five soils are the same as those in the previous embodiment, only the final results are presented here.

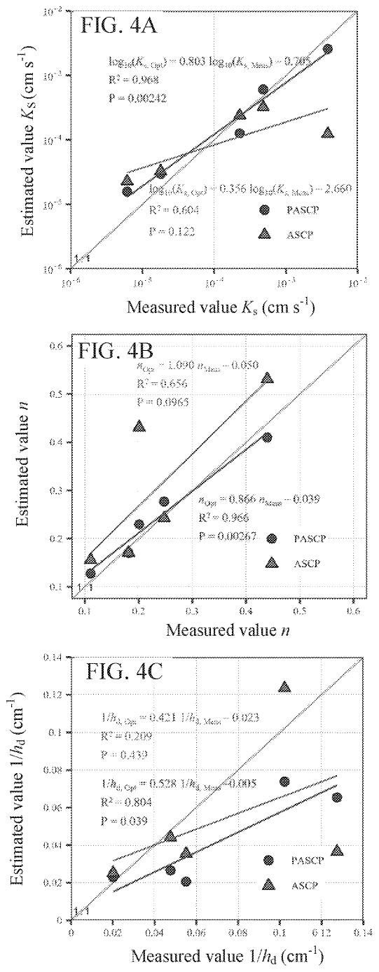

FIG. 4 shows the degree of dispersion between the parameters estimated by the PASCP and ASCP methods for the above five soils and the measured parameters. The purpose is to verify whether the PASCP method can accurately capture the parameter variation trends among different soil textures and to demonstrate the stronger robustness of the PASCP method in parameter estimation through comparison with the ASCP method. The regression analysis results show that there is a significant linear correlation (R²≥0.804, P<0.05) between the parameters (n, 1/h_{d}, and log₁₀ (Kₛ)) estimated by the PASCP method and the measured parameters, indicating that the PASCP method can effectively reflect the parameter changes caused by differences in soil texture. Among these parameters, the estimated values of n and log₁₀(Kₛ) are closer to the measured values (Slope_n=0.866, Slope_log₁₀(Kₛ)=0.803), while the estimated value of 1/h_{d} has a certain deviation, which may be related to the hysteresis effect present in the measured SWRC. In contrast, under the condition of measurement errors, the parameter estimation accuracy of the ASCP method decreases significantly, and the correlation and statistical significance between the estimated and measured parameter values are also significantly reduced. This further highlights the better stability and reliability of the PASCP method under the interference of measurement errors.

In summary, the PASCP method can not only effectively capture the parameter variation patterns among different soil textures but also possess excellent robustness and reliability even in the presence of actual measurement errors.

TABLE 1
Properties of the five soils

| | | | | P_{b} | | | θₛ | | |
|---|---|---|---|---|---|---|---|---|---|
| Soil | | Silt | | g | SOC | | -cm³ | | l* |
| type | Sand | -%- | Clay | cm⁻³ | % | θᵣ | cm⁻³⁻ | θᵢ | cm |
| Sandy | 77.3 | 12.0 | 10.7 | 1.70 | 0.11 | 0.012 | 0.315 | 0.012 | 1.395 |
| loam | | | | | | | | | |
| Loam | 30.5 | 45.3 | 24.2 | 1.50 | 0.20 | 0.029 | 0.419 | 0.029 | 1.745 |
| _1 | | | | | | | | | |
| Loam | 43.3 | 39.1 | 17.6 | 1.40 | 0.27 | 0.016 | 0.382 | 0.016 | 1.704 |
| _2 | | | | | | | | | |
| Clay | 24.5 | 41.56 | 33.9 | 1.40 | 2.08 | 0.056 | 0.460 | 0.056 | 1.876 |
| loam | | | | | | | | | |
| Clay | 20.9 | 34.9 | 44.2 | 1.45 | 0.74 | 0.025 | 0.422 | 0.025 | 1.848 |

### Embodiment 5

This embodiment has two main purposes: first, to evaluate the impact of the soil saturation time (tₛ) introduced in the present disclosure on the accuracy of parameter estimation; and second, to compensate for the estimation deviation of parameter 1/h_{d} caused by the hysteresis effect in the measured SWRC, which might otherwise underestimate the advantages of the present disclosure in terms of parameter estimation accuracy. To this end, this embodiment uses numerical simulations based on HYDRUS-1D, selecting seven virtual soils with significantly different textures as research objects (Table 2). The properties of the seven soils include: soil residual water content (θᵣ), soil saturated water content (θₛ), soil initial water content (θᵢ), pore size distribution index n, inverse of air-entry suction 1/h_{d}, and saturated hydraulic conductivity Kₛ. The parameter inversion is repeated ten times for each soil to evaluate the dispersion of the estimated parameters. The measurement steps adopted in the simulation are consistent with those in the previous embodiment, and only the simulation results are presented here.

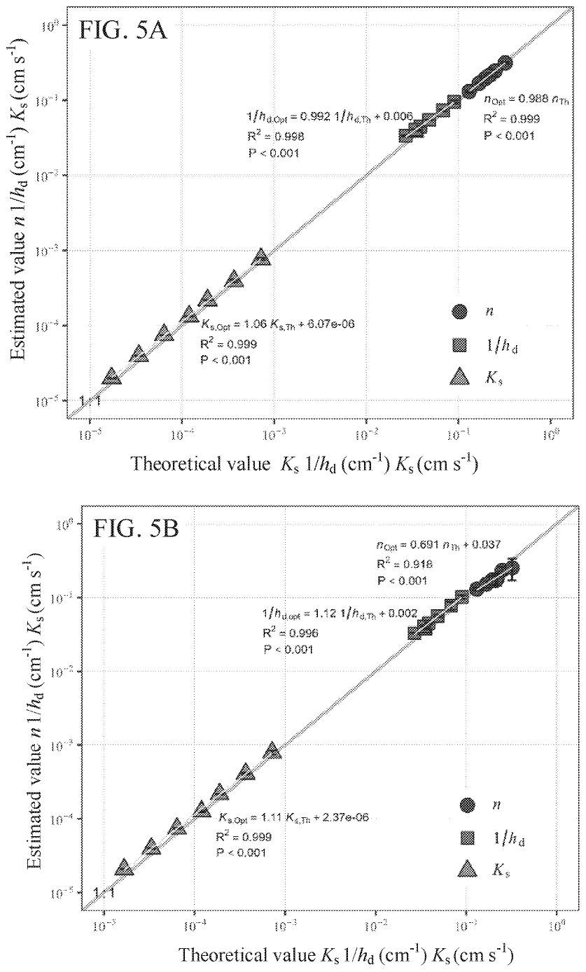

FIG. 5 shows the parameter inversion accuracy of the PASCP method for seven virtual soils with different textures. When the saturation time (tₛ) is introduced as an additional inversion constraint (FIG. 5a), the parameters (n, 1/h_{d}, and Kₛ) estimated by PASCP show a highly consistent linear relationship with the theoretical values (R²>0.99, P<0.001), where the estimated value of n is almost completely consistent with the theoretical value. Although 1/ha and Kₛ are slightly overestimated, the deviation shows a systematic trend, and the PASCP method is still able to accurately reflect the variation patterns of 1/h_{d} and Kₛ among different soil textures (R²≥0.998). Furthermore, the standard deviations of all inverted parameters are low, indicating that this method can uniquely estimate the hydraulic parameters. In contrast, without introducing the saturation time (tₛ) as an inversion constraint (FIG. 5b), n is significantly underestimated (Slope=0.691), the estimation accuracy decreases and the standard deviation increases significantly, while the prediction accuracy of 1/h_{d} and Kₛ also decreases. This result indicates that introducing the saturation time (tₛ) as an inversion constraint plays an important role in improving the accuracy and uniqueness of parameter estimation. In summary, the algorithm proposed by the present disclosure can accurately and uniquely estimate the soil hydraulic parameters.

TABLE 2
Properties of the seven soils

| | | θᵢ | | | | |
|---|---|---|---|---|---|---|
| Soil | | -cm³ | | | 1/h_{d} | Kₛ |
| type | θᵣ | cm⁻³- | θₛ | n | cm⁻¹ | cm s⁻¹ |
| Sandy | 0.041 | 0.075 | 0.453 | 0.322 | 0.0682 | 7.19e−04 |
| loam | | | | | | |
| Sandy | 0.068 | 0.101 | 0.398 | 0.250 | 0.0356 | 1.19e−04 |
| clay | | | | | | |
| loam | | | | | | |
| Loam | 0.027 | 0.071 | 0.463 | 0.220 | 0.0897 | 3.66e−04 |
| Silt | 0.015 | 0.064 | 0.501 | 0.211 | 0.0482 | 1.89e−04 |
| loam | | | | | | |
| Clay | 0.075 | 0.104 | 0.366 | 0.194 | 0.0386 | 6.39e−05 |
| loam | | | | | | |
| Loamy | 0.109 | 0.010 | 0.430 | 0.168 | 0.0343 | 3.33e−05 |
| clay | | | | | | |
| Clay | 0.090 | 0.109 | 0.475 | 0.131 | 0.0268 | 1.67e−05 |

What is claimed is:

1. A robust calculation method for soil hydraulic parameters based on a one-dimensional upward infiltration experiment, comprising following steps:
step A: applying a constant lower-boundary water head hₚ to a cutting ring soil sample, conducting a one-dimensional upward infiltration experiment on a homogeneous soil, and recording a relationship between a cumulative infiltration I of the cutting ring soil sample and a time t, a time t* when a wetting front first reaches a surface of the cutting ring soil sample, and a cumulative infiltration I_{end} when an infiltration curve tends to stabilize;
step B: calculating a saturated volumetric water content θₛ of the soil based on an initial volumetric water content θᵢ of the cutting ring soil sample, a height L* of a cutting ring, and the cumulative infiltration I_{end} when the infiltration curve tends to stabilize:
$${θ}_{s} = {θ}_{i} + \frac{{I}_{end}}{{L}^{*}} ; \qquad ( 13 )$$
step C: determining, based on the relationship between the cumulative infiltration I and the time t obtained in the step A, a single-point cumulative infiltration I* at the time t* when the wetting front first reaches the surface of the cutting ring soil sample; and
step D: obtaining, by an interval-constrained parameter optimization-based inversion method, parameters to be estimated: n, 1/h_{d}, and Kₛ, according to the saturated volumetric water content θₛ, the initial volumetric water content θᵢ, and a residual volumetric water content θᵣ of the cutting ring soil sample, the single-point cumulative infiltration I* when the wetting front first reaches the soil surface, and observed data I_{obs}(tᵢ) of the cumulative infiltration over the time;
wherein in the step D, an objective function for the interval-constrained parameter optimization-based inversion method is constructed according to Eq. (14):
$$minimize Φ = \sqrt{\frac{1}{N} {\sum }_{i = 1}^{N} {( {I}_{o b s} ( {t}_{i} ) - {I}_{sim} ( {t}_{i} ; n , 1 / {h}_{d} , {K}_{s} ) )}^{2}} + σ max \{ 0 , \vert {I}_{sim} ( {L}^{*} ; n , 1 / {h}_{d} , {K}_{s} ) - {I}^{*} \vert - ε \} , \qquad ( 14 )$$
wherein I_{obs}(tᵢ) is a measured value obtained in the step A, representing the cumulative infiltration I at an observation time point tᵢ; Iₛᵢₘ(tᵢ; n, 1/h_{d}, Kₛ) denotes a simulated cumulative infiltration at the observation time point tᵢ; and Iₛᵢₘ(L*; n, 1/h_{d}, Kₛ) denotes a cumulative infiltration obtained by simulation calculation.

2. The robust calculation method for the soil hydraulic parameters according to claim 1, wherein Iₛᵢₘ(tᵢ; n, 1/h_{d}, Kₛ) is calculated as follows:
(1) a soil saturation time tₛ is expressed as:
$${t}_{s} = ( {θ}_{s} - {θ}_{i} ) \{ - \frac{{z}_{s}^{*}}{{K}_{s}} + [ \frac{{H}_{f}}{{K}_{e}} - \frac{{h}_{d} + {h}_{p}}{{K}_{s}} ] ln ( 1 - \frac{{z}_{s}^{*}}{{h}_{d} + {h}_{p}} ) - \frac{{H}_{f}}{{K}_{e}} ln ( 1 - \frac{{K}_{s} - {K}_{e}}{{K}_{s} ( {h}_{d} + {h}_{p} )} {z}_{s}^{*} ) + \frac{{H}_{f}}{{K}_{s} - {K}_{e}} ( \frac{1}{1 + \frac{{K}_{s} - {K}_{e}}{{K}_{s} ( {h}_{d} + {h}_{p} )} {z}_{s}^{*}} - 1 ) \} \qquad ( 6 )$$
wherein
$${z}_{s}^{*}$$
denotes a functional expression of an equivalent wetting front z_{fe}, and when the equivalent wetting front z_{fe} reaches the soil surface, i.e., z_{fe}=L*, the soil reaches a saturated state, and
$${z}_{s}^{*}$$
is explicitly expressed as:
$${z}_{s}^{*} = \frac{{A}_{3} {L}^{*} + {A}_{4} + 1 - \sqrt{{( {A}_{3} {L}^{*} + {A}_{4} + 1 )}^{2} - 4 {A}_{3} {L}^{*}}}{2 {A}_{3}} \qquad ( 7 )$$
(2) when the infiltration time t is less than tₛ, a functional relationship between the cumulative infiltration I of the soil and the time tis:
$$\frac{t}{{θ}_{s} - {θ}_{i}} = - \frac{{h}_{d} + {h}_{p}}{{J}_{w 0} + {K}_{s}} + [ \frac{{H}_{f}}{{K}_{e}} - \frac{{h}_{d} + {h}_{p}}{{K}_{s}} ] ln ( 1 - \frac{{K}_{s}}{{J}_{w 0} + {K}_{s}} ) - \frac{{H}_{f}}{{K}_{e}} ln ( 1 - \frac{{K}_{s} - {K}_{e}}{{J}_{w 0} + {K}_{s}} ) + \frac{{H}_{f}}{{K}_{s} - {K}_{e}} ( \frac{1}{1 - \frac{{K}_{s} - {K}_{e}}{{J}_{w 0} + {K}_{s}}} - 1 ) \qquad ( 8 )$$
$$I = ( {θ}_{s} - {θ}_{i} ) [ \frac{{K}_{s} ( {h}_{d} + {h}_{p} )}{{J}_{w 0} + {K}_{s}} - \frac{{K}_{e} {H}_{f}}{{J}_{w 0} + {K}_{e}} ] \qquad ( 9 )$$
(3) when t is not less than tₛ, the soil reaches complete saturation, and the cumulative infiltration remains unchanged, and is calculated as follows:
I=(θₛ−θᵢ)L* (10);
substituting the parameters to be estimated, n, 1/h_{d}, Kₛ, into the equation enables a root mean square error (RMSE) between the measured value I_{obs}(tᵢ) and the simulated value Iₛᵢₘ(tᵢ; n, 1/h_{d}, Kₛ) to be minimized;
Iₛᵢₘ(L*; n, 1/h_{d}, Kₛ) is calculated as follows:
$$I = {A}_{0} {z}_{f} + \frac{( {θ}_{s} - {θ}_{i} - {A}_{0} )}{2 {A}_{1}} ( {A}_{1} {z}_{f} + {A}_{2} + 1 - \sqrt{{( {A}_{1} {z}_{f} + {A}_{2} + 1 )}^{2} - 4 {A}_{1} {z}_{f}} ) \qquad ( 11 )$$
wherein z_{f}=L*; σ denotes a penalty factor; and e denotes a constraint threshold (mm), with a value depending on an upper limit of a maximum measurement error in actual observation; and
remaining parameters are expressed as follows:
$$\{ \begin{matrix}{K}_{e} = ζ {K}_{s} = \frac{n ( a + 1 ) ( {θ}_{s} - {θ}_{i} )}{[ a ( 3 n + 2 ) + n ] [ {θ}_{s} - {θ}_{i} - a ( {θ}_{i} - {θ}_{r} ) ]} {K}_{s} \\ {H}_{f} = - \frac{1}{ζ ( 3 n + 1 )} {h}_{d} \\ {A}_{0} = ( {θ}_{s} - {θ}_{r} ) \frac{1 - ( 1 + a b ) {S}_{ei}}{b ( a + 1 )} \\ {A}_{1} = \frac{{K}_{s} - {K}_{e}}{{h}_{d} + {h}_{p}} \\ {A}_{2} = - \frac{{θ}_{s} - {θ}_{i}}{{A}_{0}} \frac{{K}_{e} {H}_{f}}{{K}_{s} ( {h}_{d} + {h}_{p} )} \\ {A}_{3} = - \frac{{K}_{s} - {K}_{e}}{{K}_{s} ( {h}_{d} + {h}_{p} )} \\ {A}_{4} = - \frac{{K}_{e} {H}_{f}}{{K}_{s} ( {h}_{d} + {h}_{p} )}\end{matrix} ; \qquad ( 12 )$$
wherein parameters a and b are defined as follows:
$$a = \frac{n ( 2 n + 3 )}{( 2 n + 2 ) ( 2 n + 1 )} \qquad ( 2 )$$
$$b = 1 - {S}_{ei}^{\frac{1}{a}} \qquad ( 3 )$$
wherein Sₑᵢ denotes an effective saturation corresponding to the initial volumetric water content θᵢ.

3. The robust calculation method for the soil hydraulic parameters according to claim 2, wherein Eq. (14) is solved using a genetic algorithm (GA) in R language: the algorithm generates 1000 sets of parameters to be estimated, n, 1/h_{d}, Kₛ, in each generation; the algorithm stops when φ in Eq. (14) does not improve for 500 consecutive generations or when a cumulative number of iterations reaches 5000; a local search strategy is enabled, wherein parameter search ranges are: Kₛ=[10⁻⁶, 10⁻²] cm·s⁻¹, 1/h_{d}=[0.01, 1] cm⁻¹, and n=[0.1, 0.7]; logarithmic transformation is performed on Kₛ and 1/h_{d}; the constraint threshold ε is set to 0.4 mm; and σ is set to \sqrt{.Machine$double.xmax}, wherein .Machine$double.xmax denotes a maximum double-precision floating-point number representable in R language.
